(19) United States
(12) Patent Application Publication
(10) Pub. No.: US 2019/0128884 A1
(43) Pub. Date: May 2, 2019

(54) MARKER SEQUENCES FOR MANAGING THE THERAPY OF RHEUMATOID ARTHRITIS PATIENTS

(71) Applicant: PROTAGEN AG, Dortmund (DE)

(72) Inventors: Petra BUDDE, Dortmund (DE); Angelika LÜKING, Bochum (DE); Peter SCHULZ-KNAPPE, Hemmingen (DE); Hans-Dieter ZUCHT, Hannover (DE)

(21) Appl. No.: 16/310,147

(22) PCT Filed: Jun. 15, 2017
(86) PCT No.: PCT/EP2017/064728

§ 371 Date: Dec. 14, 2018

(30) Foreign Application Priority Data
Jun. 15, 2016 (EP) 16174672.2

(51) Int. Cl. G01N 33/564; C07K 16/24

(52) U.S. Cl. CPC G01N 33/564; C07K 16/24; A61K 31/52; G01N 2800/52; G01N 2800/102

(57) ABSTRACT

The present invention relates to a method for managing the therapy of patients and patient (sub)populations (responder/non-responder) suffering from rheumatoid arthritis (RA) and to the use of suitable marker sequences, in particular in the form of panels, diagnostic agents and test kits, and to their use in rheumatoid arthritis (RA) diagnosis, prognosis and therapy management, in particular for drug-based therapy.

The present invention relates to a method for managing the therapy of patients and patient (sub)populations (responder/non-responder) suffering from rheumatoid arthritis (RA) and to the use of suitable marker sequences, in particular in the form of panels, diagnostic agents and test kits, and to their use in rheumatoid arthritis (RA) diagnosis, prognosis and therapy management, in particular for drug-based therapy.

Rheumatoid arthritis (RA) is an autoimmune disease affecting approximately 1% of the population of the western world. Chronic inflammatory processes within the first year of the disease (early RA) lead already to progressive, joint-destroying synovitis. If the RA is not identified in good time and treated aggressively within a certain time window (“window of opportunity”), it leads to the destruction of joints with significant physical limitations and systematic manifestations, which lead to disabilities and reduced life expectancy.

In the early phase of the disease, for patients with joint inflammation, the criteria for diagnosis of RA and differentiation between arthritis and merely arthralgia (joint pain) are often difficult. This delays the therapy management for patients with possible early RA, and therefore the joint destruction can progress if the diagnosis is unclear.

Markers for the early detection of RA or prognosis and therapy management are therefore immensely important, in particular in patients of rheumatoid arthritis (RA) who are already receiving drug-based therapy.

The current classification criteria published jointly by the American College for Rheumatology (ACR) and the European League Against Rheumatism (EULAR) in 2010 (Aletaha, Neogi et al. 2010) are intended to facilitate an early diagnosis of RA by the determination of autoantibodies (AAB) against citrullinated antigens (ACPAs), such that disease-modifying therapy is started as early as possible and irreversible consequences of the disease are prevented.

ACPA autoantibodies are positively detectable in approximately 75% of patients with established RA, but only in approximately 62% of patients with early RA.

This emphasises the need for further markers for the early diagnosis of RA, prognosis, and therapy management.

Marker sequences for the diagnosis of RA are disclosed in WO2009030226. These marker sequences were discovered by a method in which serum samples from RA patients and those from healthy individuals were examined comparatively and the results were analysed statistically. The marker sequences described in WO 2009030226 are suitable for the diagnosis of RA, but are not sufficiently suitable for predicting the therapy response.

Previously, RA was considered to be a disease in which primarily autoantibodies against citrullinated peptides are produced. In a small number of publications, autoantibodies against posttranslational unmodified proteins or peptides have also been described (Hueber, Tomooka et al. 2009; Somers, Geusens et al. 2011), however there have been no attempts to seek new autoantibodies systematically or to use these for the identification of patient (sub)groups.

Based on the recommendations published in 2010 of the European League Against Rheumatism (EULAR), the primary goal of the treatment of RA is to stop the disease progression and achieve disease remission (Smolen, Landewé et al. 2010).

The prospect of success of disease remission can be significantly improved, in particular in early RA, by the use of a disease-modifying therapy (DMARD—disease modifying anti-rheumatic drug, or what is known as basic therapy).

Conventional DMARDs include a group of drugs which has disease-modifying properties going beyond symptomatic effects.

The effect of DMARDs is generally delayed, with the time until response being 4-16 weeks.

In particular methotrexate (MTX), which belongs to the group of conventional DMARDs, has become part of the standard and initial therapy for RA. Other suitable drugs are azathioprine, sulfasalazine, chloroquine/hydroxychloroquine, leflunomide, cyclophosphamide, D-penicillamine or cyclosporine and also biological DMARDs.

The so-called biological DMARDs include therapeutic antibodies, which block the effect of the inflammatory cytokine—tumor necrosis factor alpha (TNF). TNF is a central regulator of the immune system and is involved in inflammatory processes in a series of rheumatoid diseases, such as RA, lupus, ankylosing spondylitis, psoriasis, Crohn's disease, colitis ulcerosa and juvenile chronic arthritis.

The development of what are known as TNF blockers has significantly improved the treatment of RA and other rheumatoid diseases and has led to a significant improvement in quality of life.

Generally, there are two approaches for inhibiting TNF activity. On the one hand, by means of monoclonal therapeutic antibodies, the binding of TNF to its receptor can be prevented. To this end, various anti-TNF blockers are approved for the treatment of RA, such as infliximab (Remicade®), adalimumab (Humira®), certolizumab pegol (Cimzia®) and golimumab (Simponi®).

It is also possible to inhibit the binding of TNF to the body's own receptor by soluble, recombinantly produced TNF receptor, such as etanercept (Enbrel®).

In spite of generally good efficacy, merely 40-50% of RA patients demonstrate a satisfactory clinical improvement under MTX therapy. Furthermore, MTX therapy may be accompanied by toxic effects and undesirable side-effects, which often cause patients to abort MTX therapy.

In numerous clinical studies it has been possible to show that the various TNF blockers demonstrate a comparable efficacy compared to MTX and largely inhibit the progression of RA with damage of the joints. Clinical studies, however, have shown that approximately 20-50% of the treated RA patients respond only insufficiently to TNF blockers. Alternative therapies have been developed for these patients and are based for example on the inhibition of B-cells, such as rituximab (Rituxan).

In view of the high treatment costs and possible side-effects, however, there are no suitable predictors, that is to say diagnostic marker sequences, for the selection of specific and suitable therapy forms and also for clinical decision-making for RA patients.

By means of the use according to the invention of markers (marker sequences) for identifying patients that respond to MTX therapy (responder=R) or that do not respond (non-responder=NR), it is possible to select the best therapy for patient (sub)groups having a specific marker profile.

In accordance with EULAR and ACR recommendations, firstly conventional DMARDs (usually MTX) are administered following diagnosis, and the clinical efficacy is monitored over six months. In the absence of efficacy or in the event that side-effects are encountered, it is possible to change to a biological DMARD, usually a TNF blocker. This has the disadvantage, however, that in the case of an absence of efficacy, valuable time is lost and the disease continues to progress unhindered.

Clinical studies have shown that early intense treatment of patients with early RA using combination therapy constituted by TNF blockers and MTX can also improve the prognosis of the disease compared to MTX monotherapy (Detert, Bastian et al. 2013) and represents an alternative drug-based therapy.

There is an absence, however, of suitable predictors or markers (marker sequences) for identifying (sub)groups of patients who might benefit from aggressive combination therapy of this kind.

The object of the present invention is therefore to improve the therapy by means of drug-based treatment (DMARD) and also in respect of clinical decision-making.

The object is achieved by the technical teaching of the claims.

The object of the invention is therefore also to provide a prognosis and to manage therapy in the treatment of RA patients by means of an in-vitro diagnosis, in particular for RA patients who are already undergoing drug-based therapy.

The object is achieved in accordance with the invention in that a method for in-vitro diagnosis is provided, which contains selected autoantigens (markers) for detecting autoantibodies from bodily fluids of an RA patient undergoing drug-based therapy.

The invention therefore relates to a method for stratification and therapy management of rheumatoid arthritis patients undergoing drug-based therapy, wherein responders and/or non-responders to the drug-based therapy are identified by means of an in-vitro diagnosis, wherein at least one marker sequence is selected from the group SEQ ID No. 1-208.

The autoantigens or markers (marker sequences) according to the invention are identified by means of a differential method comprising multiple steps, in which serum samples from healthy individuals and RA patients undergoing drug-based therapy are examined comparatively in respect of their reactivity with multiple potential antigens, and these results are statistically analysed.

The selection of the serum samples and the sequence of the steps surprisingly make it possible to identify markers (marker sequences) in patients with early and established RA undergoing drug-based therapy and are also suitable for identifying RA patient (sub)groups and can increase the prospect of success to therapeutic success.

The invention therefore relates to a method for identifying marker sequences for rheumatoid arthritis (RA), comprising the steps of:
a) bringing serum samples of RA patients into contact with more than 5,000 antigens coupled to (Luminex) beads, measuring the binding of the individual antigens to proteins in the serum of the RA patients by immunofluorescence assay, and determining the median fluorescence intensity (MFI) for each individual antigen;
b) bringing serum samples of healthy individuals into contact with the same antigens coupled to (Luminex) beads, measuring the binding of the individual antigens to proteins in the serum of the healthy individuals by immunofluorescence assay, and determining the median fluorescence intensity (MFI) for each individual antigen;
c) statistically evaluating the MFI data of each individual antigen from a) and b) by means of univariant analysis and thus identifying markers with which RA patients can be distinguished from healthy individuals;
d) wherein the markers are selected from the sequences SEQ ID No. 1 to 208, partial sequences or fragments thereof, and homologues of sequences SEQ ID No. 1-208 with at least 90% homology.

In the field of microarrays flat substrates are used, to which marker sequences or sequences to be examined are bound. In protein biochips the marker sequences to be examined or the sequences binding to these marker sequences are immobilised on a solid, flat support. An alternative arrangement or panel of marker sequences or sequences to be examined is possible on beads, which therefore differ inter alia in view of their sensitivity and specificity from conventional microarrays. Bead arrays are created for example by impregnating pellets either with different concentrations of fluorescent dye or for example by barcode technology. The pellets can be addressed and can be used to identify specific binding events that occur on their surface. Bead technology is based on microscopically small spherical pellets or platelets, which are referred to as microspheres or beads. These beads can serve analogously to ELISA and Western Blot as solid phase for biochemical detection reactions. A wide range of different bead types are available, which for example differ in their fluorescence shade and each of which carries its own specific detection reagent on the surface. In this way, an accordingly large number of different detection reactions can be carried out simultaneously in a very small sample volume. With bead arrays specific interactions between two defined biochemical compounds can be detected. Compared with conventional microarrays, the bead-based validation is characterised by a particularly high sensitivity and specificity. With the method according to the invention and the use of beads for validation, marker sequences for RA can be identified that differ in terms of their sensitivity and specificity from the previously known marker sequences.

The invention also relates to a marker sequence for rheumatoid arthritis obtainable by a method according to the invention, wherein the marker sequence is selected from the group of sequences SEQ ID No. 1 to 208, partial sequences or fragments thereof, and homologues of sequences SEQ ID No. 1-208 with at least 90% homology.

The invention also relates to the use of one or more marker sequence(s) according to the invention for the stratification and therapy management of rheumatoid arthritis A further embodiment concerns the use according to the invention, characterised in that 2 or 3, preferably 4 or 5, particularly preferably 6, 7 or 8 or more different marker sequences, for example 10 to 20 or 30 or more different marker sequences, are used. A set of marker sequences of this kind is referred to as a panel.

One embodiment concerns the use according to the invention, characterised in that the marker sequence(s) is/are applied to a solid support, wherein the solid support is selected from filters, membranes, wafers, for example silicon wafers, glass, metal, plastic, chips, mass spectrometry targets, matrices, and beads, for example magnetic, coated or labelled beads, such as fluorophore-labelled beads or Luminex beads.

The invention also relates to a method for the stratification and therapy management of rheumatoid arthritis, wherein

a.) at least one marker sequence according to the invention from SEQ ID No. 1-208 is applied to a solid support, preferably to a bead and

b.) is brought into contact with bodily fluid or tissue sample of a patient and

c.) an interaction of the bodily fluid or of the tissue sample with the marker sequence from a.) is detected.

Such an interaction can be detected for example by a probe, in particular by an antibody.

The invention also relates to a method for stratification, in particular for risk stratification, or for therapy management of a patient with rheumatoid arthritis, wherein at least one marker sequence according to the invention is used in order to examine a sample from the patient.

One embodiment concerns a method according to the invention for diagnosing rheumatoid arthritis, wherein the stratification or the therapy management includes decisions regarding the treatment and therapy of the patient, in particular the hospitalisation of the patient, the use, efficacy and/or dosage of one or more drugs, a therapeutic measure or the monitoring of the course of the disease and the course of therapy, aetiology or classification of the disease, inclusive of prognosis.

The invention also relates to an arrangement or panel comprising or consisting of one or more marker sequence(s) according to the invention. Preferred panels are the subject matter of the examples.

The invention also relates to an assay or protein array comprising an arrangement or panel according to the invention.

The invention also relates to a diagnostic agent for the diagnosis of rheumatoid arthritis containing at least one marker sequence according to the invention and where appropriate further auxiliaries and additives.

The invention therefore relates to the use of marker sequences for the stratification or therapy management of rheumatoid arthritis, wherein at least one marker sequence selected from the group of marker sequences SEQ ID No. 1-208 and/or the genomic sequences comprising one of the sequences SEQ ID No. 1-104 and/or a protein coded by the sequences SEQ ID No. 105-208, partial sequences or fragments thereof, and homologues of sequences SEQ ID No. 1-208 with at least 90% homology is determined for a patient to be examined.

A further embodiment of the invention concerns the use of the marker sequence(s) according to the invention for the stratification or therapy management of rheumatoid arthritis, characterised in that the determination is performed by means of in-vitro diagnosis.

The invention therefore relates to a method for identifying early RA patients undergoing drug-based therapy who do not respond to conventional DMARDs, in particular MRX, and may be non-responders or therapy-resistant.

These RA markers according to the invention are the subject of SEQ ID No. 1-3 or 105-107 of the antigens in Table 1 and Table 2, which can be used for the stratification or therapy management of RA and predict or diagnose whether an RA patient will respond to MTX treatment. Marker candidate antigens which have a p-value for the non-parametric mean value comparison between groups of <0.05, but at the same time are reactive in RA patients were selected on the basis of the univariant results for the generation of these markers.

The identified markers CCDC136 (SEQ ID No. 1), HSPB1 (SEQ ID No. 2), and IGFBP2 (SEQ ID No. 3) are therefore preferred in accordance with the invention for determining a resistance to drug-based therapy, in particular a resistance to MTX therapy.

A further embodiment of the invention relates to a method for identifying markers for identifying patient (sub)groups that respond or do not respond to conventional DMARD therapy, such as MTX.

Markers which were found by means of this embodiment of the method are named for example in Table 1 in group 5 and relate to SEQ ID No. 4-51 and/or SEQ ID No. 108-155.

A further embodiment of the invention relates to a method for identifying markers for identifying patient (sub)groups that respond or do not respond to combination therapy of biological and conventional DMARD, such as anti-TNF blockers and MTX.

Markers which were found by means of this embodiment of the method are named for example in Table 1 in group 6 and relate to SEQ ID No. 52-104 and/or SEQ ID No. 156-208.

On account of the high clinical and serological heterogeneity of the RA disease, it is difficult to provide an unequivocal RA diagnosis using just one biomarker. It is therefore often necessary to combine maximum uncorrelated autoantigens to form what are known as panels of markers (biomarker panels for RA).

For example, within the scope of tailored medicine, corresponding panels of markers for RA can be individually compiled for individual patients or patient groups for the relevant RA subtype (subgroup). It is therefore also necessary to provide multiple potential markers for RA in order to select appropriate RA markers specific to corresponding subgroups or subtypes for each individual case. A corresponding panel can be provided for example in the form of an arrangement, an array or also one or more beads, preferably Luminex beads. The invention thus relates to an arrangement comprising one or more markers according to the invention, a protein array comprising one or more markers according to the invention, a bead (pellet or platelet) comprising one or more markers according to the invention.

The marker sequences according to the invention were able to be identified by means of differential screening of samples from healthy test subjects with patient samples with rheumatoid arthritis. The marker sequences according to the invention were then expressed and, following coupling of the expressed marker sequence candidates to Luminex beads, validated with the aid of the Luminex beads, partly by comparison with known biomarkers for rheumatoid arthritis. Highly specific marker sequences could thus be identified for rheumatoid arthritis.

“Beads” (pearls, pellets, originally also referred to as latex particles) designate what are known as microspheres or microparticles, which are used as supports for biomolecules in tests and assays. Uniform (approximately equally sized) microparticles that are produced by special chemical methods are required for tests and assays. These methods are known to a person skilled in the art.

Beads for different applications are also commercially available (for example from the company Progen Biotechnik GmbH). Beads may consist of different materials, for example glass, polystyrene, PMMA and different other polymers, partly also copolymers. Beads can be labelled with different dyes or dye mixtures and can be provided with coatings. Biomolecules can be coupled to the surface of beads. Different coupling methods are available for this purpose and are known to a person skilled in the art, for example adsorption or covalent coupling. The surface of the beads can be modified, such that a directed coupling of the biomolecules on the bead surface, for example in conjunction with spacers, tags or special modifications, is possible, and whereby the analytical sensitivity can be further increased.

The term “rheumatoid arthritis (RA)” is defined for example by Pschyrembel, de Gruyter, 261^{st} edition (2007), Berlin. In accordance with the invention “juvenile idiopathic arthritis” is also included (ICD-10: M08.-. abb.: JIA. Earlier synonyms: juvenile rheumatoid arthritis, juvenile chronic arthritis, Still's disease or the popular name “child's rheumatism”) and is the collective term for a series of diseases primarily affecting the joints (arthritis) of rheumatic origin in childhood (juvenile) (definition for example according to Pschyrembel, de Gruyter, 261^{st} edition (2007), Berlin).

In a preferred embodiment the marker sequences are determined outside the human body and the determination is performed in an ex vivo/in vitro diagnosis.

In the sense of this invention, “diagnosis” means the positive determination of rheumatoid arthritis by means of the marker sequences according to the invention as well as the assignment of the patients to the indication “rheumatoid arthritis”. The term diagnosis includes the medical diagnostics and examinations in this regard, in particular in-vitro diagnostics and laboratory diagnostics, and also proteomics and nucleic acid blotting. Further tests may be necessary to be sure and to exclude other diseases. The term diagnosis therefore also includes the differential diagnosis of rheumatoid arthritis by means of the marker sequences according to the invention, and the prognosis in the case of determined rheumatoid arthritis.

The invention also relates to a method for the stratification, in particular risk stratification and/or therapy management of a patient with rheumatoid arthritis, for example in a patient with a very early stage of RA that cannot be detected by means of the marker CCP, wherein at least one marker sequence according to the invention is determined on a patient to be examined. The stratification of the patient with rheumatoid arthritis in new or established sub-groups within the disease rheumatoid arthritis is also included, as well as the expedient selection of patient groups for the selection for therapy with certain active agents. The term therapy management also includes, in accordance with the invention, the division of patients into responders and non-responders in respect of a drug-based therapy or the course of a therapy.

In accordance with the invention “stratification or therapy management” means in particular the prediction/prognosis and monitoring of the response or non-response to a medicament/drug for the treatment of RA or a modified therapy and also follow-up care.

In the sense of this invention, “stratification or therapy management” means that the method according to the invention renders possible decisions for the treatment and therapy of the patient, whether it is the hospitalisation of the patient, the use, efficacy and/or dosage of one or more drugs, a therapeutic measure, or the monitoring of the course of a disease and the course of therapy or aetiology or classification of RA, for example into a new or existing subtype, or the differentiation of RA and relevant patients.

In a further embodiment of the invention, the term “stratification” in particular includes the risk stratification with the prognosis of an “outcome” of a negative health event.

Within the scope of this invention, the term “patient” is understood to mean any test subject (human or mammal), with the provision that the test subject is examined for rheumatoid arthritis, in particular therapeutic success or therapeutic improvement, or with the provision that the test subject or the individual is examined for RA and is receiving or being administered drugs for the treatment of RA, for example by means of basic therapy (see above).

The term “marker sequences” in the sense of this invention means that the nucleic acid sequence, for example the mRNA, cDNA or the polypeptide or protein obtainable therefrom are significant for rheumatoid arthritis. By way of example the mRNA or cDNA or the polypeptide or protein obtainable therefrom can interact with substances from the bodily fluid or tissue sample of a patient with rheumatoid arthritis (for example (auto)antigen (epitope)/(auto)antibody (paratope) interaction). In a particularly preferred embodiment of the invention the RA marker is an antigen or part of an antigen or codes for an antigen or for part of an antigen.

In the sense of the invention “wherein at least one marker sequence selected from the group of marker sequences SEQ ID No. 1-208 and/or the genomic sequences comprising one of the sequences SEQ ID No. 1-104 and/or a protein coded by the sequences SEQ ID No. 105-208, partial sequences or fragments thereof, or homologues of sequences SEQ ID No. 1-208 with at least 90% homology is determined for a patient to be examined” means that an interaction between the bodily fluid or the tissue sample of a patient and the marker sequence(s) according to the invention is detected. Such an interaction is, for example, a binding, in particular a binding substance at least at one of the marker sequences according to the invention, or in the case of a cDNA is the hybridisation with a suitable substance under selected conditions, in particular stringent conditions (for example as defined typically in J. Sambrook, E. F. Fritsch, T. Maniatis (1989), Molecular cloning: A laboratory manual, 2nd Edition, Cold Spring Habor Laboratory Press, Cold Spring Habor, USA or Ausubel, “Current Protocols in Molecular Biology”, Green Publishing Associates and Wiley Interscience, N.Y. (1989)). One example for stringent hybridisation conditions is: hybridisation in 4×SSC at 65° C. (alternatively in 50% formamide and 4×SSC at 42° C.), followed by a number of washing steps in 0.1×SSC at 65° C. for a total of about one hour. One example for less stringent hybridisation conditions is hybridisation in 4×SSC at 37° C., followed by a number of washing steps in 1×SSC at room temperature.

Such substances, in accordance with the invention, are part of a bodily fluid, in particular blood, whole blood, blood plasma, blood serum, patient serum, urine, cerebrospinal fluid, synovial fluid, or a tissue sample of the patient.

The substances from the bodily fluid or tissue sample occur or are expressed either only, or at least in an intensified manner in the case of RA, whereas these substances are not present or at least are present only to a smaller extent (smaller amount, lower concentration) in patients without RA or healthy individuals. RA markers can also be characterised on the other hand in that they interact with substances from the bodily fluid or tissue sample of RA patients because these substances are no longer expressed or are expressed at least in a much smaller amount/concentration in the case of RA, whereas these substances are present or are present at least in a much higher level in patients without RA. Markers for RA can also be provided in healthy test subjects, but their amount (concentration) changes for example with the emergence, establishment and therapy of RA. One or more markers can in this way depict a profile of substances from bodily fluid and tissue sample, for example an RA-associated autoantibody profile of the patient in question. Markers according to the invention are biomarkers for RA.

Autoantibody profiles comprise the amount of one or more autoantibodies, the occurrence/expression of which is accompanied by the emergence and/or establishment of RA. Autoantibody profiles thus comprise on the one hand the composition, i.e. for example one or more autoantibodies is/are expressed only in the case of RA, and on the other hand the amount/concentration of individual autoantibodies, i.e. the amount/concentration of individual autoantibodies changes with the emergence and establishment of RA. These changes can be detected with the aid of the marker sequences according to the invention.

The marker sequences according to the invention SEQ ID No. 1-208 are specified in Table 1 and can be unambiguously identified (see RefSeq Accession or GI Accession) by the respective cited database entries (also by means of the Internet: http://www.ncbi.nlm.nih.gov/).

The invention therefore also relates to the full-length sequences of the marker sequences according to the invention and the marker sequences as defined in the tables via the known database entries and also the marker sequences specified in the accompanying sequence protocol.

The invention furthermore likewise includes analogous embodiments of the marker sequences, in particular of the nucleic acid sequences SEQ ID No. 1-104 and the protein sequences SEQ ID No. 105-208.

In a further embodiment of the invention marker sequences are preferred that have P-values less than or equal to 0.006, preferably less than or equal to 0.001 or less than or equal to 0.0001, particularly preferably less than or equal to 0.00001 (see the tables).

In a further embodiment of the invention homologues of the marker sequences according to the invention are included. In particular, these are homologues having an identity of 70%, 80% or 85%, preferably 90%, 91%, 92%, 93%, 94% or 95% identity, in particular 96%, 97%, 98%, 99% or more identity, with the marker sequences according to the invention and suitable for the use according to the invention—the detection of rheumatoid arthritis (what are known as “homologues” or homologous marker sequences). Homologues can be protein sequences or nucleic acid sequences.

Partial sequences or fragments are sequences that comprise 50 to 100 nucleotides or amino acids, preferably 70-120 nucleotides or amino acids, particularly preferably 100 to 200 nucleotides or amino acids of one of the marker sequences SEQ ID No. 1-208.

In accordance with the invention the marker sequences also comprise modifications of the nucleotide sequence, for example of the cDNA sequence and the corresponding amino acid sequence, such as chemical modification, for example citrullination, acetylation, phosphorylation, glycosylation or polyA strand and further modifications known accordingly to a person skilled in the art.

In a further embodiment the respective marker sequence can be represented in different amounts in one or more regions on a solid support, for example a bead. This allows a variation of the sensitivity. The regions may each comprise a totality of marker sequences, i.e. a sufficient number of different marker sequences, in particular 2 to 5 or 10 or more marker sequences, and where appropriate further nucleic acids and/or proteins, in particular biomarkers. However, at least 96 to 25,000 (numerically) or more different or identical marker sequences and further nucleic acids and/or proteins, in particular biomarkers, are preferred. Furthermore, more than 2,500 different or identical marker sequences are preferred, particularly preferably 10,000 or more, and where appropriate further nucleic acids and/or proteins, in particular biomarkers.

Within the scope of this invention, “arrangement” or “panel” is synonymous with “array”, and, if this “array” is used to identify substances to be bound on marker sequences, this is to be understood to be an “assay” or a diagnostic device. In a preferred embodiment the arrangement is designed such that the marker sequences represented on the arrangement are present in the form of a grid on a solid support. Furthermore, preferred arrangements are those that permit a high-density arrangement of marker sequences, and the marker sequences are spotted.

Such high-density spotted arrangements are disclosed for example in WO 99/57311 and WO 99/57312 and can be used advantageously in a robot-assisted automated high-throughput method.

Within the scope of this invention, however, the term “assay” or diagnostic device likewise comprises those embodiments of a device such as ELISA (for example individual wells of a microtitre plate are coated with the marker sequences or combinations of marker sequences according to the invention, and where appropriate are applied to the individual wells of the microtitre plate in a robot-assisted manner; examples include diagnostic ELISA kits from the company Phadia or “Searchlight” Multiplex ELISA kits from the company Pierce/Thermo Fisher Scientific), bead-based assay (spectrally distinguishable bead populations are coated with marker sequences/combinations of marker sequences. The patient sample is incubated with this bead population and bound (auto)antibodies are detected by means of a further fluorescence-labelled secondary antibody or a detection reagent by measuring the fluorescence; for example Borrelia IgG kit or Athena Multilyte from the company Multimetrix), line assay (marker sequences or combinations of marker sequences according to the invention are immobilised in a robot-assisted manner on membranes, which are examined or incubated with the patient sample; example “Euroline” from the company Euroimmun AG), Western Blot (example “Euroline-WB” from the company Euroimmun AG), and immunochromatographic methods (for example what are known as lateral flow immunoassays; marker sequences or combinations of marker sequences are immobilised on test strips (membranes, U.S. Pat. No. 5,714,389 and many others); example “One Step HBsAg” test device from Acon Laboratories) or similar immunological single or multiplex detection methods.

A further object of the invention is therefore that of providing a diagnostic device or an assay, in particular a protein biochip, which allows a stratification and therapy management for rheumatoid arthritis.

In order to achieve this object, the marker sequences of the arrangement or panel according to the invention are fixed on a solid support, but preferably spotted or immobilised or imprinted, i.e. are applied reproducibly. One or more marker sequences can be present repeatedly in the totality of all marker sequences and can be present in different amounts based on a spot. Furthermore, the marker sequences on the solid support can be standardised (for example by means of serial dilution series for example of human globulins as internal calibrators for data standardisation and quantitative assessment).

The invention therefore concerns an assay or protein biochip or one or more beads (bead-based assay) consisting of an arrangement or panel containing marker sequences according to the invention.

In a further embodiment the marker sequences are present as clones. Such clones can be obtained for example by means of a cDNA expression library according to the invention (Bissow et al. 1998 (above)). In a preferred embodiment such expression libraries containing clones are obtained using expression vectors from a cDNA expression library consisting of the cDNA marker sequences. These expression vectors preferably contain inducible promoters. The induction of the expression can be carried out for example by means of an inducer, such as IPTG. Suitable expression vectors are described in Terpe et al. (Terpe T Appl Microbiol Biotechnol. 2003 January; 60(5):523-33).

Expression libraries are known to a person skilled in the art; they can be produced in accordance with standard works, such as Sambrook et al, “Molecular Cloning, A laboratory handbook, 2nd edition (1989), CSH press, Cold Spring Harbor, N.Y. Expression libraries that are tissue-specific (for example human tissue, in particular human organs) are furthermore preferable. Further, expression libraries that can be obtained by means of exon-trapping are also comprised in accordance with the invention. Instead of the term expression library, reference may also be made synonymously to an expression bank.

Protein biochips or beads or corresponding expression libraries that do not exhibit any redundancy (what is known as a Uniclone® library) and that can be produced in accordance with the teaching of WO 99/57311 and WO 99/57312 are furthermore preferred. These preferred Uniclone® libraries have a high proportion of non-defective fully expressed proteins of a cDNA expression library.

Within the scope of this invention the clones can also be, but are not limited to, transformed bacteria, recombinant phages or transformed cells of mammals, insects, fungi, yeasts or plants.

The clones are fixed, spotted or immobilised on a solid support.

The invention therefore relates to an arrangement, wherein the marker sequences are present as clones.

In addition, the marker sequences can be present in the respective form of a fusion protein, which for example contains at least one affinity epitope or “tag”. The tag may be or may contain one such as c-myc, his tag, arg tag, FLAG, alkaline phosphatase, V5 tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

A marker sequence can be composed of a number of individual marker sequences. This may include the cloning of individual fragments to form a large common fragment and the expression of this combined fragment.

In all embodiments, the term “solid support” includes embodiments such as a filter, a membrane, a magnetic or fluorophore-labelled pellet, a silicon wafer, glass, metal, plastic, a chip, a mass spectrometry target or a matrix.

However, a filter and beads are preferred in accordance with the invention.

Furthermore, PVDF, nitrocellulose or nylon is preferred as a filter (for example Immobilon P Millipore, Protran Whatman, Hybond N+ Amersham).

In a further preferred embodiment of the arrangement according to the invention, this corresponds to a grid with the dimensions of a microtiter plate (8-12 well strips, 96 wells, 384 wells or more), a silicon wafer, a chip, a mass spectrometry target or a matrix.

In a further preferred embodiment pellets or what are known as beads are used as support. Here, bead-based multiplex assays are preferably used. The analysis and evaluation of the bead-based assays can be performed for example with a Luminex analysis system, which is performed on the basis of the method of flow cytometry with use of two different lasers.

Whereas the measurements on planar protein arrays offer merely a dynamic range of 1.5-2 magnitudes (powers of 10), a dynamic range of 3.5-4 magnitudes can be covered by the use of Luminex beads. The measurements in the low response ranges also provide very good coefficients of variation (CVs), i.e. no more than 10%.

Whereas the measurements on planar protein arrays offer merely coefficients of variation (CVs) from 10 to 25% (intra-array comparison) or 10 to 50% (inter-array comparison), the CVs of the Luminex measurements are located between 3 to 10%. An assay quality not generally achieved by commercial ELISAs is thus provided. The known disadvantages (limited plexing rate by interference of different detection antibodies) for Luminex-based analysis and diagnostic methods do not occur with the UNIarray concept, since merely a single fluorescence-labelled anti-human IgG from goat, sheep or mouse is used as detection probe. Due to the transfer of the UNIarray concept to Luminex (i.e. bead-based protein arrays), a number of apparatuses can additionally be saved, i.e. protein printers, hybridisation machines and array readers, and can be replaced by one apparatus. Here, the UNIarray concept is not bound to Luminex, but can also be used on other platforms, such as Randox, VBC Genomics, etc. The high measurement accuracy and the low CVs of the individual measurements allow the use of better and new statistical methods for the identification of potent individual markers and also for rapid sorting of false positives.

Protein-protein interactions (for example protein at the marker sequence, such as antigen/antibody) or corresponding “means for detecting the binding success” can be visualised for example by means of fluorescence labelling, biotinylation, radio-isotope labelling or colloidal gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc., and the corresponding colorimetric, fluorescent or chemiluminescent substrates. A readout is performed for example by means of a microarray laser scanner, a CCD camera or visually.

### EXAMPLES

TABLE 1
Marker sequences (antigens)

| No. | Gene ID | Gene Symbol | Gene Name | Group |
|---|---|---|---|---|
| 1 | 64753 | CCDC136 | coiled-coil domain containing 136 | Group 1 |
| 2 | 3281 | HSBP1 | heat shock factor binding protein 1 | Group 1 |
| 3 | 3485 | IGFBP2 | insulin-like growth factor binding protein | Group 1 |
| | | | 2, 36 kDa | |
| 4 | 10476 | ATP5H | ATP synthase, H+ transporting, | Group 5 |
| | | | mitochondrial Fo complex, subunit d | |
| 5 | 7812 | CSDE1 | cold shock domain containing E1, RNA- | Group 5 |
| | | | binding | |
| 6 | 1485 | CTAG1B | cancer/testis antigen 1B | Group 5 |
| 7 | 8320 | EOMES | eomesodermin | Group 5 |
| 8 | 10961 | ERP29 | endoplasmic reticulum protein 29 | Group 5 |
| 9 | 84893 | FBXO18 | F-box protein, helicase, 18 | Group 5 |
| 10 | 3059 | HCLS1 | hematopoietic cell-specific Lyn substrate 1 | Group 5 |
| 11 | 3182 | HNRNPAB | heterogeneous nuclear ribonucleoprotein A/B | Group 5 |
| 12 | 3798 | KIF5A | kinesin family member 5A | Group 5 |
| 13 | 8079 | MLF2 | myeloid leukemia factor 2 | Group 5 |
| 14 | 60560 | NAA35 | N(alpha)-acetyltransferase 35, NatC | Group 5 |
| | | | auxiliary subunit | |
| 15 | 8473 | OGT | O-linked N-acetylglucosamine (GlcNAc) | Group 5 |
| | | | transferase | |
| 16 | 5174 | PDZK1 | PDZ domain containing 1 | Group 5 |
| 17 | 23645 | PPP1R15A | protein phosphatase 1, regulatory subunit 15A | Group 5 |
| 18 | 64062 | RBM26 | RNA binding motif protein 26 | Group 5 |
| 19 | 23170 | TTLL12 | tubulin tyrosine ligase-like family member 12 | Group 5 |
| 20 | 91544 | UBXN11 | UBX domain protein 11 | Group 5 |
| 21 | 6944 | VPS72 | vacuolar protein sorting 72 homolog | Group 5 |
| | | | (S. cerevisiae) | |
| 22 | 163033 | ZNF579 | zinc finger protein 579 | Group 5 |
| 23 | 23625 | FAM89B | family with sequence similarity 89, member B | Group 5 |
| 24 | 27344 | PCSK1N | proprotein convertase subtilisin/kexin | Group 5 |
| | | | type 1 inhibitor | |
| 25 | 64762 | GAREM | GRB2 associated, regulator of MAPK1 | Group 5 |
| 26 | 30 | ACAA1 | acetyl-CoA acyltransferase 1 | Group 5 |
| 27 | 11093 | ADAMTS13 | ADAM metallopeptidase with thrombospondin | Group 5 |
| | | | type 1 motif, 13 | |
| 28 | 204 | AK2 | adenylate kinase 2 | Group 5 |
| 29 | 211 | ALAS1 | 5′-aminolevulinate synthase 1 | Group 5 |
| 30 | 90416 | C15orf57 | chromosome 15 open reading frame 57 | Group 5 |
| 31 | 51531 | TRMO | tRNA methyltransferase O | Group 5 |
| 32 | 1108 | CHD4 | chromodomain helicase DNA binding protein 4 | Group 5 |
| 33 | 22982 | DIP2C | disco-interacting protein 2 homolog C | Group 5 |
| 34 | 9454 | HOMER3 | homer scaffolding protein 3 | Group 5 |
| 35 | 3490 | IGFBP7 | insulin-like growth factor binding protein 7 | Group 5 |
| 36 | 10445 | MCRS1 | microspherule protein 1 | Group 5 |
| 37 | 112950 | MED8 | mediator complex subunit 8 | Group 5 |
| 38 | 65003 | MRPL11 | mitochondrial ribosomal protein L11 | Group 5 |
| 39 | 7469 | NELFA | negative elongation factor complex member A | Group 5 |
| 40 | 84081 | NSRP1 | nuclear speckle splicing regulatory | Group 5 |
| | | | protein 1 | |
| 41 | 81572 | PDRG1 | p53 and DNA-damage regulated 1 | Group 5 |
| 42 | 5494 | PPM1A | protein phosphatase, Mg2+/Mn2+ dependent, 1A | Group 5 |
| 43 | 5536 | PPP5C | protein phosphatase 5, catalytic subunit | Group 5 |
| 44 | 9727 | RAB11FIP3 | RAB11 family interacting protein 3 (class II) | Group 5 |
| 45 | 10111 | RAD50 | RAD50 homolog, double strand break repair | Group 5 |
| | | | protein | |
| 46 | 6449 | SGTA | small glutamine-rich tetratricopeptide | Group 5 |
| | | | repeat (TPR)-containing, alpha | |
| 47 | 116841 | SNAP47 | synaptosomal-associated protein, 47 kDa | Group 5 |
| 48 | 10290 | SPEG | SPEG complex locus | Group 5 |
| 49 | 10422 | UBAC1 | UBA domain containing 1 | Group 5 |
| 50 | 65109 | UPF3B | UPF3 regulator of nonsense transcripts | Group 5 |
| | | | homolog B (yeast) | |
| 51 | 8882 | ZPR1 | ZPR1 zinc finger | Group 5 |
| 52 | 39 | ACAT2 | acetyl-CoA acetyltransferase 2 | Group 6 |
| 53 | 348 | APOE | apolipoprotein E | Group 6 |
| 54 | 128061 | C1orf131 | chromosome 1 open reading frame 131 | Group 6 |
| 55 | 79714 | CCDC51 | coiled-coil domain containing 51 | Group 6 |
| 56 | 6249 | CLIP1 | CAP-GLY domain containing linker protein 1 | Group 6 |
| 57 | 30827 | CXXC1 | CXXC finger protein 1 | Group 6 |
| 58 | 9704 | DHX34 | DEAH (Asp-Glu-Ala-His) box polypeptide 34 | Group 6 |
| 59 | 57572 | DOCK6 | dedicator of cytokinesis 6 | Group 6 |
| 60 | 84444 | DOT1L | DOT1-like histone H3K79 methyltransferase | Group 6 |
| 61 | 23301 | EHBP1 | EH domain binding protein 1 | Group 6 |
| 62 | 8661 | EIF3A | eukaryotic translation initiation factor | Group 6 |
| | | | 3, subunit A | |
| 63 | 1982 | EIF4G2 | eukaryotic translation initiation factor 4 | Group 6 |
| | | | gamma, 2 | |
| 64 | 63877 | FAM204A | family with sequence similarity 204, | Group 6 |
| | | | member A | |
| 65 | 54865 | GPATCH4 | G patch domain containing 4 | Group 6 |
| 66 | 2922 | GRP | gastrin-releasing peptide | Group 6 |
| 67 | 84064 | HDHD2 | haloacid dehalogenase-like hydrolase | Group 6 |
| | | | domain containing 2 | |
| 68 | 80895 | ILKAP | integrin-linked kinase-associated | Group 6 |
| | | | serine/threonine phosphatase | |
| 69 | 23479 | ISCU | iron-sulfur cluster assembly enzyme | Group 6 |
| 70 | 57498 | KIDINS220 | kinase D-interacting substrate, 220 kDa | Group 6 |
| 71 | 3875 | KRT18 | keratin 18, type I | Group 6 |
| 72 | 29094 | LGALSL | lectin, galactoside-binding-like | Group 6 |
| 73 | 79888 | LPCAT1 | lysophosphatidylcholine acyltransferase 1 | Group 6 |
| 74 | 4357 | MPST | mercaptopyruvate sulfurtransferase | Group 6 |
| 75 | 10528 | NOP56 | NOP56 ribonucleoprotein | Group 6 |
| 76 | 51602 | NOP58 | NOP58 ribonucleoprotein | Group 6 |
| 77 | 9315 | NREP | neuronal regeneration related protein | Group 6 |
| 78 | 23762 | OSBP2 | oxysterol binding protein 2 | Group 6 |
| 79 | 55593 | OTUD5 | OTU deubiquitinase 5 | Group 6 |
| 80 | 79668 | PARP8 | poly (ADP-ribose) polymerase family, | Group 6 |
| | | | member 8 | |
| 81 | 23089 | PEG10 | paternally expressed 10 | Group 6 |
| 82 | 9360 | PPIG | peptidylprolyl isomerase G (cyclophilin G) | Group 6 |
| 83 | 84687 | PPP1R9B | protein phosphatase 1, regulatory subunit 9B | Group 6 |
| 84 | 5589 | PRKCSH | protein kinase C substrate 80K-H | Group 6 |
| 85 | 5834 | PYGB | phosphorylase, glycogen; brain | Group 6 |
| 86 | 117584 | RFFL | ring finger and FYVE-like domain | Group 6 |
| | | | containing E3 ubiquitin protein ligase | |
| 87 | 9025 | RNF8 | ring finger protein 8, E3 ubiquitin | Group 6 |
| | | | protein ligase | |
| 88 | 6091 | ROBO1 | roundabout guidance receptor 1 | Group 6 |
| 89 | 6240 | RRM1 | ribonucleotide reductase M1 | Group 6 |
| 90 | 22937 | SCAP | SREBF chaperone | Group 6 |
| 91 | 23256 | SCFD1 | sec1 family domain containing 1 | Group 6 |
| 92 | 6382 | SDC1 | syndecan 1 | Group 6 |
| 93 | 10483 | SEC23B | Sec23 homolog B, COPII coat complex | Group 6 |
| | | | component | |
| 94 | 6576 | SLC25A1 | solute carrier family 25 (mitochondrial | Group 6 |
| | | | carrier; citrate transporter), member 1 | |
| 95 | 23384 | SPECC1L | sperm antigen with calponin homology and | Group 6 |
| | | | coiled-coil domains 1-like | |
| 96 | 23635 | SSBP2 | single-stranded DNA binding protein 2 | Group 6 |
| 97 | 6612 | SUMO3 | small ubiquitin-like modifier 3 | Group 6 |
| 98 | 84260 | TCHP | trichoplein, keratin filament binding | Group 6 |
| 99 | 9804 | TOMM20 | translocase of outer mitochondrial | Group 6 |
| | | | membrane 20 homolog (yeast) | |
| 100 | 55850 | USE1 | unconventional SNARE in the ER 1 homolog | Group 6 |
| | | | (S. cerevisiae) | |
| 101 | 89891 | WDR34 | WD repeat domain 34 | Group 6 |
| 102 | 54521 | WDR44 | WD repeat domain 44 | Group 6 |
| 103 | 23036 | ZNF292 | zinc finger protein 292 | Group 6 |
| 104 | 6234 | RPS28 | ribosomal protein S28 | Group 6 |

TABLE 2
3 Markers for early diagnosis of RA and
identification of MTX therapy failure

| | | | | early RA (HitHard) | | |
|---|---|---|---|---|---|---|
| | | | Gene | | Fold- | Sens. (%) at |
| No. | Group | gene ID | Symbol | p-value | change | 90% spec. |
| 1 | Group 1 | 64753 | CCDC136 | 0.031 | 1.7 | 14 |
| 2 | Group 1 | 3281 | HSBP1 | 0.002 | 1.3 | 25 |
| 3 | Group 1 | 3485 | IGFBP2 | 0.0004 | 1.5 | 19 |

Table 2 contains 3 antigens which are particularly suitable for identifying RA patients who are ACPA-negative and likely will not benefit from MTX basic therapy.

TABLE 3
51 markers for controlling MTX therapy

| | | | | PLAC/ | PLAC/ | Therapy |
|---|---|---|---|---|---|---|
| | | | Gene | MTX | MTX fold | manage- |
| No. | Group | GeneID | Symbol | p-value | change | ment |
| 1 | Group 1 | 64753 | CCDC136 | 0.050 | −5.15 | NR |
| 2 | Group 1 | 3281 | HSBP1 | 0.004 | −2.15 | NR |
| 3 | Group 1 | 3485 | IGFBP2 | 0.025 | −1.65 | NR |
| 4 | Group 5 | 10476 | ATP5H | 0.021 | −2.14 | NR |
| 5 | Group 5 | 7812 | CSDE1 | 0.037 | −1.96 | NR |
| 6 | Group 5 | 1485 | CTAG1B | 0.013 | −1.54 | NR |
| 7 | Group 5 | 8320 | EOMES | 0.023 | −2.58 | NR |
| 8 | Group 5 | 10961 | ERP29 | 0.018 | −1.57 | NR |
| 9 | Group 5 | 84893 | FBXO18 | 0.006 | −1.50 | NR |
| 10 | Group 5 | 3059 | HCLS1 | 0.019 | −3.02 | NR |
| 11 | Group 5 | 3182 | HNRNPAB | 0.001 | −2.43 | NR |
| 12 | Group 5 | 3798 | KIF5A | 0.003 | −1.76 | NR |
| 13 | Group 5 | 8079 | MLF2 | 0.042 | −1.58 | NR |
| 14 | Group 5 | 60560 | MAK10 | 0.018 | −1.54 | NR |
| 15 | Group 5 | 8473 | OGT | 0.025 | −1.55 | NR |
| 16 | Group 5 | 5174 | PDZK1 | 0.023 | −2.24 | NR |
| 17 | Group 5 | 23645 | PPP1R15A | 0.007 | −1.82 | NR |
| 18 | Group 5 | 64062 | RBM26 | 0.016 | −2.13 | NR |
| 19 | Group 5 | 23170 | TTLL12 | 0.020 | −2.92 | NR |
| 20 | Group 5 | 91544 | UBXN11 | 0.004 | −1.77 | NR |
| 21 | Group 5 | 6944 | VPS72 | 0.030 | −1.87 | NR |
| 22 | Group 5 | 163033 | ZNF579 | 0.016 | −2.09 | NR |
| 23 | Group 5 | 23625 | FAM89B | 0.013 | −1.70 | NR |
| 24 | Group 5 | 27344 | PCSK1N | 0.005 | −2.99 | NR |
| 25 | Group 5 | 64762 | FAM59A | 0.026 | −3.45 | NR |
| 26 | Group 5 | 30 | ACAA1 | 0.034 | 2.59 | R |
| 27 | Group 5 | 11093 | ADAMTS13 | 0.011 | 1.61 | R |
| 28 | Group 5 | 204 | AK2 | 0.025 | 1.87 | R |
| 29 | Group 5 | 211 | ALAS1 | 0.007 | 3.64 | R |
| 30 | Group 5 | 90416 | C15orf57 | 0.031 | 1.69 | R |
| 31 | Group 5 | 51531 | C9orf156 | 0.008 | 1.69 | R |
| 32 | Group 5 | 1108 | CHD4 | 0.028 | 1.95 | R |
| 33 | Group 5 | 22982 | DIP2C | 0.012 | 1.58 | R |
| 34 | Group 5 | 9454 | HOMER3 | 0.019 | 1.87 | R |
| 35 | Group 5 | 3490 | IGFBP7 | 0.021 | 1.98 | R |
| 36 | Group 5 | 10445 | MCRS1 | 0.015 | 1.54 | R |
| 37 | Group 5 | 112950 | MED8 | 0.031 | 1.91 | R |
| 38 | Group 5 | 65003 | MRPL11 | 0.003 | 4.15 | R |
| 39 | Group 5 | 7469 | WHSC2 | 0.035 | 1.73 | R |
| 40 | Group 5 | 84081 | CCDC55 | 0.039 | 1.82 | R |
| 41 | Group 5 | 81572 | PDRG1 | 0.013 | 2.16 | R |
| 42 | Group 5 | 5494 | PPM1A | 0.039 | 1.75 | R |
| 43 | Group 5 | 5536 | PPP5C | 0.005 | 1.98 | R |
| 44 | Group 5 | 9727 | RAB11FIP3 | 0.033 | 1.81 | R |
| 45 | Group 5 | 10111 | RAD50 | 0.012 | 2.55 | R |
| 46 | Group 5 | 6449 | SGTA | 0.004 | 2.49 | R |
| 47 | Group 5 | 116841 | SNAP47 | 0.041 | 1.59 | R |
| 48 | Group 5 | 10290 | SPEG | 0.013 | 2.15 | R |
| 49 | Group 5 | 10422 | UBAC1 | 0.007 | 2.16 | R |
| 50 | Group 5 | 65109 | UPF3B | 0.020 | 2.13 | R |
| 51 | Group 5 | 8882 | ZNF259 | 0.021 | 1.56 | R |

NR: marker increased in the case of MTX therapy resistance (therapy non-responder)
R: marker increased in the case of MTX therapy response (therapy responder patients)

TABLE 4
53 markers for controlling anti-TNF inhibitor therapy

| | | | | | ADA/ | ADA/ |
|---|---|---|---|---|---|---|
| | | | | ADA/ | MTX | MTX |
| | | | Gene | MTX | fold- | therapy |
| No. | Group | GeneID | Symbol | p-value | change | management |
| 52 | Group 6 | 39 | ACAT2 | 0.029 | −1.80 | NR |
| 53 | Group 6 | 348 | APOE | 0.020 | 1.90 | R |
| 54 | Group 6 | 128061 | C1orf131 | 0.013 | 2.16 | R |
| 55 | Group 6 | 79714 | CCDC51 | 0.042 | −2.36 | NR |
| 56 | Group 6 | 6249 | CLIP1 | 0.036 | −1.53 | NR |
| 57 | Group 6 | 30827 | CXXC1 | 0.020 | 1.58 | R |
| 58 | Group 6 | 9704 | DHX34 | 0.010 | −1.77 | NR |
| 59 | Group 6 | 57572 | DOCK6 | 0.011 | −1.69 | NR |
| 60 | Group 6 | 84444 | DOT1L | 0.010 | −2.04 | NR |
| 61 | Group 6 | 23301 | EHBP1 | 0.031 | 1.62 | R |
| 62 | Group 6 | 8661 | EIF3A | 0.009 | 1.71 | R |
| 63 | Group 6 | 1982 | EIF4G2 | 0.039 | −2.20 | NR |
| 64 | Group 6 | 63877 | C10orf84 | 0.020 | −2.01 | NR |
| 65 | Group 6 | 54865 | GPATCH4 | 0.003 | 2.36 | R |
| 66 | Group 6 | 2922 | GRP | 0.039 | −1.80 | NR |
| 67 | Group 6 | 84064 | HDHD2 | 0.010 | −1.65 | NR |
| 68 | Group 6 | 80895 | ILKAP | 0.018 | 1.61 | R |
| 69 | Group 6 | 23479 | ISCU | 0.040 | 1.58 | R |
| 70 | Group 6 | 57498 | KIDINS220 | 0.011 | −1.72 | NR |
| 71 | Group 6 | 3875 | KRT18 | 0.018 | −1.63 | NR |
| 72 | Group 6 | 29094 | HSPC159 | 0.011 | −1.94 | NR |
| 73 | Group 6 | 79888 | LPCAT1 | 0.025 | −2.10 | NR |
| 74 | Group 6 | 4357 | MPST | 0.002 | 2.94 | R |
| 75 | Group 6 | 10528 | NOP56 | 0.034 | 1.50 | R |
| 76 | Group 6 | 51602 | NOP58 | 0.000 | 2.21 | R |
| 77 | Group 6 | 9315 | C5orf13 | 0.004 | 1.60 | R |
| 78 | Group 6 | 23762 | OSBP2 | 0.021 | −1.87 | NR |
| 79 | Group 6 | 55593 | OTUD5 | 0.021 | −1.74 | NR |
| 80 | Group 6 | 79668 | PARP8 | 0.015 | −1.95 | NR |
| 81 | Group 6 | 23089 | PEG10 | 0.016 | 2.09 | R |
| 82 | Group 6 | 9360 | PPIG | 0.035 | 1.66 | R |
| 83 | Group 6 | 84687 | PPP1R9B | 0.044 | 1.73 | R |
| 84 | Group 6 | 5589 | PRKCSH | 0.005 | −2.54 | NR |
| 85 | Group 6 | 5834 | PYGB | 0.021 | −1.57 | NR |
| 86 | Group 6 | 117584 | RFFL | 0.014 | −1.94 | NR |
| 87 | Group 6 | 9025 | RNF8 | 0.032 | −1.54 | NR |
| 88 | Group 6 | 6091 | ROBO1 | 0.005 | 2.14 | R |
| 89 | Group 6 | 6240 | RRM1 | 0.023 | −1.68 | NR |
| 90 | Group 6 | 22937 | SCAP | 0.041 | −1.64 | NR |
| 91 | Group 6 | 23256 | SCFD1 | 0.005 | 2.39 | R |
| 92 | Group 6 | 6382 | SDC1 | 0.007 | −2.15 | NR |
| 93 | Group 6 | 10483 | SEC23B | 0.018 | −2.13 | NR |
| 94 | Group 6 | 6576 | SLC25A1 | 0.031 | −3.58 | NR |
| 95 | Group 6 | 92521 | CYTSB | 0.017 | −2.91 | NR |
| 96 | Group 6 | 23635 | SSBP2 | 0.021 | −2.44 | NR |
| 97 | Group 6 | 6612 | SUMO3 | 0.014 | −1.62 | NR |
| 98 | Group 6 | 84260 | TCHP | 0.026 | −1.68 | NR |
| 99 | Group 6 | 9804 | TOMM20 | 0.021 | 1.97 | R |
| 100 | Group 6 | 55850 | USE1 | 0.046 | −1.64 | NR |
| 101 | Group 6 | 89891 | WDR34 | 0.019 | −1.71 | NR |
| 102 | Group 6 | 54521 | WDR44 | 0.007 | 2.18 | R |
| 103 | Group 6 | 23036 | ZNF292 | 0.003 | 2.85 | R |
| 104 | Group 6 | 6234 | RPS28 | 0.028 | −1.55 | NR |

NR: marker increased in the case of ADA/MTX combination therapy resistance (therapy non-responder)
R: marker increased in the case of ADA/MTX combination therapy response (therapy responder patients)

### EXAMPLES

### Example 1: Modelling for Discrimination of Therapy Responders and Non-Responders by Means of PLS

Based on EULAR/ACR guidelines for the treatment of RA patients, the main objective is to achieve a remission of the RA by means of suitable therapy. This is often defined as the attainment of a DAS28 value of <2.6. If this is not possible, the lowest possible level of disease activity should be achieved.

In a first step, a partial least square (PLS) regression model was created. By means of what is known as a PLS score plot, it is possible to visualise whether it is possible with use of the selected antigens to distinguish between what are known as therapy responder patients and non-responder patients. All antigens from Table 1 were used initially for the modelling, and the autoantigen body mirror of all serum samples was analysed at the time TO before therapy was started.

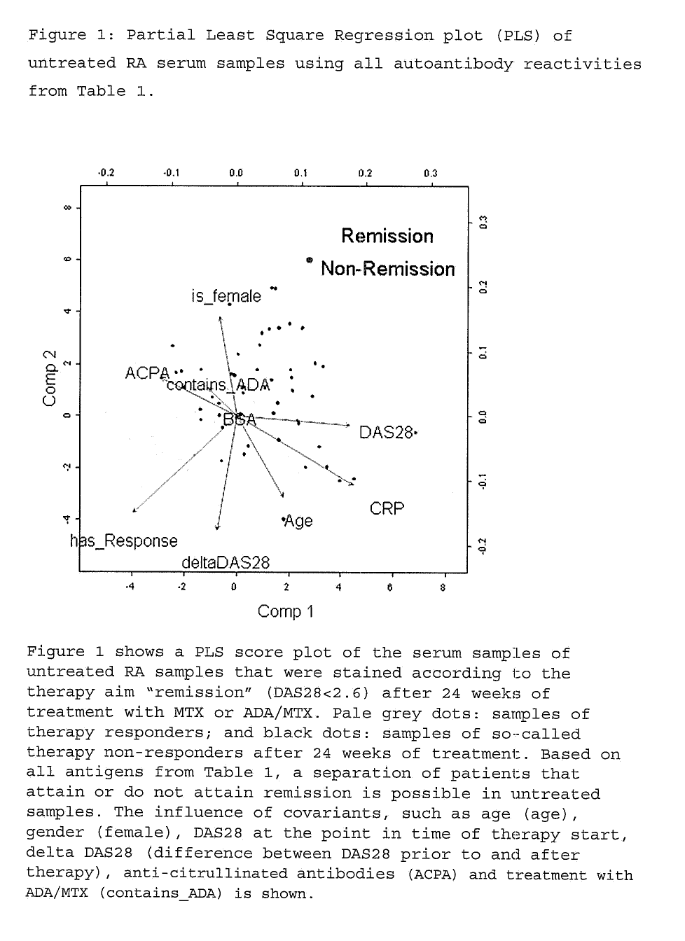

FIG. 1 shows a PLS score plot for all antigens from Table 1 and the autoantibody measured values thereof. FIG. 1 shows that the separation of patients who are reaching or missing the therapy target of remission before therapy is started is possible in principle. Antigens for supporting various therapy approaches can thus be specified from Table 1.

### Example 2: Calculation of Antigen Panels for Predicting the Response to MTX Monotherapy

On account of the clinical and serological heterogeneity of the RA disease, it is not possible with just one biomarker to predict the response to MTX therapy for all RA patients. It is therefore necessary to combine as many uncorrelated autoantigens as possible to form what are known as biomarker panels.

Group 5 of the antigens in Table 3 comprises the most important 51 antigens which are used for the calculation of biomarker panels for MTX therapy management.

Group 5 contains 26 antigens, that is to say autoantibody reactivities (antigens) which can be measured in RA patients: ACAA1, ADAMTS13, AK2, ALAS1, C15orf57, C9orf156, CHD4, DIP2C, HOMER3, IGFBP7, MCRS1, MED8, MRPL11, WHSC2, CCDC55, PDRG1, PPM1A, PPP5C, RAB11FIP3, RAD50, SGTA, SNAP47, SPEG, UBAC1, UPF3B, ZNF259. From these 26 antigens, antigens can be selected which supplement one another particularly well as markers in smaller panels. Panel 1 in Table 5 contains a combination of 11 markers which can be used selectively to identify patients who will likely respond to MTX therapy.

For the generation of panels, antigens were selected, based on the univariant results, that had a p-value for the non-parametric mean value comparison of the serum samples prior to the start of therapy between RA patients who, after 24 weeks of treatment with MTX, responded and did not respond to the therapy. A p-value of <0.05 and at the same time a different median distribution of 1.5 between the two groups were used as a basis. For the 52 antigens cited in Table 3, an L1-penalised logistic regression model was created. Antigens which were not taken into consideration within the scope of the modelling were removed from any further consideration.

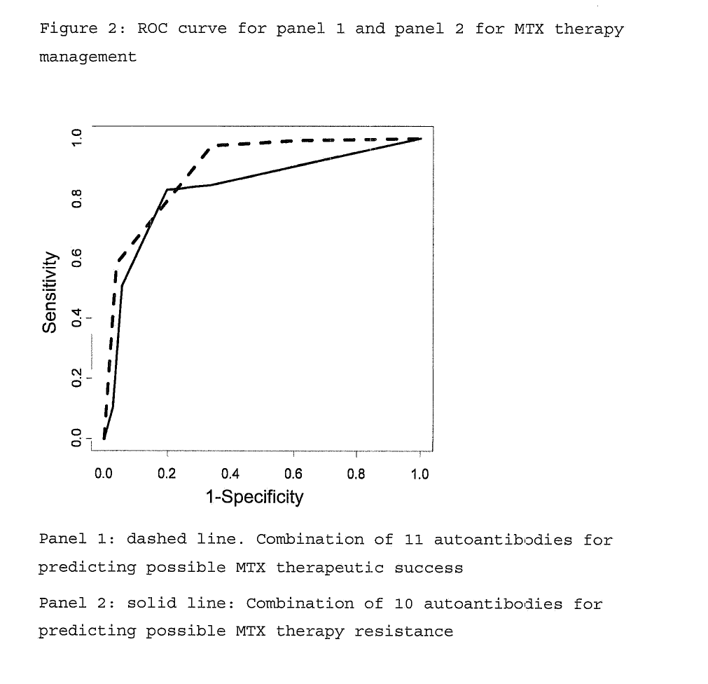

As summarised in Table 5, an AUC of 0.9 with a mean sensitivity of 98% and a mean specificity of 72% was achieved with 11 markers of panel 1. FIG. 2 shows the ROC for panel 1.

### Example 3: Calculation of Antigen Panels for Predicting MTX Therapy Resistance

Group 5 in Table 3 contains 26 autoantibody reactivities (antigens) which can be measured in RA patients who likely will not respond to MTX therapy: CCDC136, HSBP1, IGFBP2, ATP5H, CSDE1, CTAG1B, EOMES, ERP29, FBXOl8, HCLS1, HNRNPAB, KIF5A, MLF2, MAK10, OGT, PDZK1, PPP1R15A, RBM26, TTLL12, UBXN11, VPS72, ZNF579, FAM89B, PCSK1N, FAM59A.

From these 26 antigens, antigens can be selected which supplement one another particularly well as markers in smaller panels. Panel 1 in Table 5 contains a combination of 10 markers which can be used selectively to identify patients who likely will not respond to MTX therapy.

Table 5 shows different combinations of antigens which can be used for the calculation of the biomarker panels for MTX therapy management.

TABLE 5
Antigens and biomarker panels for MTX therapy-management

| Marker | Antigen | | Therapy | | | |
|---|---|---|---|---|---|---|
| No. | Symbol | Panel | management | ABC | Sens. | Spec. |
| 43 | PPP5C | 1 | MTX therapy | 0.9 | 0.89 | 0.72 |
| 38 | MRPL11 | | response | | | |
| 31 | TRMO | | | | | |
| 45 | RAD50 | | | | | |
| 27 | ADAMTS13 | | | | | |
| 33 | DIP2C | | | | | |
| 32 | CHD4 | | | | | |
| 29 | ALAS1 | | | | | |
| 51 | ZPR1 | | | | | |
| 41 | PDRG1 | | | | | |
| 37 | MED8 | | | | | |
| 11 | HNRNPAB | 2 | MTX therapy | 0.88 | 0.82 | 0.9 |
| 18 | RBM26 | | resistance | | | |
| 20 | UBXN11 | | | | | |
| 23 | FAM89B | | | | | |
| 19 | TTLL12 | | | | | |
| 17 | PPP1R15A | | | | | |
| 25 | GAREM | | | | | |
| 12 | KIF5A | | | | | |
| 10 | HCLS1 | | | | | |
| 22 | ZNF579 | | | | | |

For panel 2 it has been shown by way of example that what are known as therapy failures can be identified with a mean AUC of 0.88 and a sensitivity of 82% with a specificity of 90%.

FIG. 2 shows the sensitivity and specificity, and the area under the curve (AUC) for panel 1 and panel 2.

### Example 4: Calculation of Antigen Panels for Predicting Therapeutic Success Following Treatment with Anti-TNF Inhibitors

Group 6 in Table 4 contains 20 autoantibody responses (antigens) which can be measured in RA patients that will likely respond to anti-TNF alpha inhibitor therapy: APOE, Clorf131, CXXC1, EHBP1, EIF3A, GPATCH4, ILKAP, ISCU, MPST, NOP56, NOP58, C5orf13, PEG10, PPIG, PPP1R9B, ROBO1, SCFD1, TOMM20, WDR44, ZNF292

20 antigens can be selected from group 6 which supplement one another particularly well as markers in smaller panels. Panel 3 in Table 5 contains a combination of 11 markers which can be used selectively to identify patients that likely will not respond to MTX therapy.

Table 6 shows different combinations of antigens which can be used for the calculation of the biomarker panels for ADA/MTX therapy management.

TABLE 6
Antigens and biomarker panels
for ADA/MTX therapy-management

| Marker | Antigen | | Therapy | | | |
|---|---|---|---|---|---|---|
| No. | Symbol | Panel | management | AUC | Sens. | Spec. |
| 76 | NOP58 | 3 | ADA/MTX therapy | 0.82 | 0.79 | 0.78 |
| 91 | SCFD1 | | response | | | |
| 74 | MPST | | | | | |
| 68 | ILKAP | | | | | |
| 88 | ROBO1 | | | | | |
| 69 | ISCU | | | | | |
| 61 | EHBP1 | | | | | |
| 83 | PPP1R9B | | | | | |
| 103 | ZNF292 | | | | | |
| 99 | TOMM20 | | | | | |
| 54 | C1orf131 | | | | | |
| 95 | SPECC1L | 4 | ADA/MTX therapy | 0.95 | 0.88 | 0.89 |
| 93 | SEC23B | | resistance | | | |
| 67 | HDHD2 | | | | | |
| 56 | CLIP1 | | | | | |
| 101 | WDR34 | | | | | |
| 63 | EIF4G2 | | | | | |
| 96 | SSBP2 | | | | | |
| 90 | SCAP | | | | | |
| 85 | PYGB | | | | | |
| 59 | DOCK6 | | | | | |
| 86 | RFFL | | | | | |
| 97 | SUMO3 | | | | | |

For panel 3 it has been shown by way of example that patients who will likely respond to ADA/MTX combination therapy can be identified with a mean AUC of 0.82 and a sensitivity of 79% with a specificity of 78%.

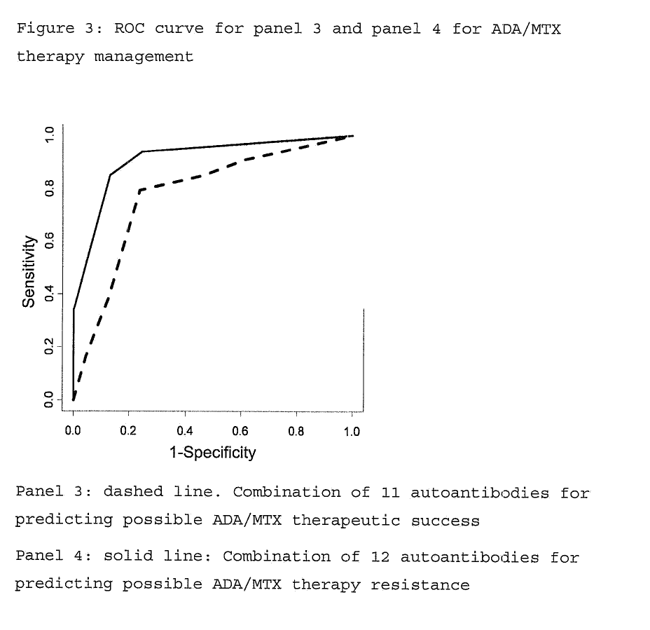

FIG. 3 shows the sensitivity and specificity, and the area under the curve (AUC) for panel 3 and panel 4.

### Example 5: Calculation of Antigen Panels for Predicting Therapy Failure Following Treatment with Anti-TNF Inhibitors

Group 6 in Table 4 contains 33 autoantibody responses (antigens) which can be measured in RA patients who likely will not respond to anti-TNF alpha inhibitor therapy: ACAT2, CCDC51, CLIP1, DHX34, DOCK6, DOT1L, EIF4G2, C10orf84, GRP, HDHD2, KIDINS220, KRT18, HSPC159, LPCAT1, OSBP2, OTUDS, PARP8, PRKCSH, PYGB, RFFL, RNF8, RRM1, SCAP, SDC1, SEC23B, SLC25A1, CYTSB, SSBP2, SUMO3, TCHP, USE1, WDR34, RPS28

33 antigens can be selected from group 6 which supplement one another particularly well as markers in smaller panels. Panel 4 in Table 6 contains a combination of 12 markers which can be used selectively to identify patients who likely will not respond to ADA/MTX therapy and therefore should be treated using an alternative biological DMARD.

For panel 4 it has been shown by way of example that what are known as ADA/MTX therapy failures can be identified with a mean AUC of 0.95 and a sensitivity of 88% with a specificity of 89%.

FIG. 3 shows the sensitivity and specificity, as well as the area under the curve (AUC) for panel 4.

### LITERATURE

Aletaha, D., T. Neogi, et al. (2010). “2010 Rheumatoid arthritis classification criteria: an American College of Rheumatology/European League Against Rheumatism collaborative initiative.” Ann Rheum Dis 69(9): 1580-1588.
Detert, J., H. Bastian, et al. (2013). “Induction therapy with adalimumab plus methotrexate for 24 weeks followed by methotrexate monotherapy up to week 48 versus methotrexate therapy alone for DMARD-naïve patients with early rheumatoid arthritis: HIT HARD, an investigator-initiated study.” Ann Rheum Dis 72(6): 844-850.
Hueber, W., B. Tomooka, et al. (2009). “Blood autoantibody and cytokine profiles predict response to anti-tumor necrosis factor therapy in rheumatoid arthritis.” Arthritis Res Ther 11(3): R76.
Smolen, J. S., R. Landewd, et al. (2010). “EULAR recommendations for the management of rheumatoid arthritis with synthetic and biological disease-modifying antirheumatic drugs.” Ann Rheum Dis 69(6): 964-975.
Somers, K., P. Geusens, et al. (2011). “Novel autoantibody markers for early and seronegative rheumatoid arthritis.” J Autoimmun 36(1): 33-46.

1-12. (canceled)

13. A method for managing the therapy of rheumatoid arthritis patients undergoing drug-based therapy, wherein responders and/or non-responders to the drug-based therapy are identified by an in-vitro diagnosis, characterised in that at least one marker sequence is selected from the group of SEQ ID NO: 1-208.

14. The method of claim 13, wherein patients resistant to the drug-based therapy are identified by an in-vitro diagnosis, characterised in that at least one marker sequence is selected from the group of SEQ ID NO: 1-3 and/or 105-107, SEQ ID NO: 4-51 and/or 108-155, SEQ ID NO: 52-104 and/or 156-208.

15. The method of claim 13, wherein the drug-based therapy comprises the drug methotrexate, azathioprine, sulfasalazine, chloroquine/hydroxychloroquine, leflunomide, cyclophosphamide, D-penicillamine or cyclosporine, characterised in that at least one marker sequence is selected from the group SEQ ID NO: 1-3 and/or 105-107, SEQ ID NO: 4-51 and/or 108-155.

16. The method of claim 15, wherein the drug-based therapy comprises the drug methotrexate.

17. The method of claim 13, wherein the drug-based therapy comprises the drugs constituted by monoclonal therapeutic proteins, TNF blockers, and anti-TNF blockers, characterised in that at least one marker sequence is selected from the group SEQ ID NO: 52-104 and/or 156-208.

18. The method of claim 17, characterised in that the therapy is implemented in combination with drugs selected from the group methotrexate, azathioprine, sulfasalazine, chloroquine/hydroxychloroquine, leflunomide, cyclophosphamide, D-penicillamine or cyclosporine.

19. The method of claim 18, wherein the drug-based therapy comprises the drug methotrexate.

20. The method of claim 13, comprising further clinical decisions, such as altering the therapy, aborting the therapy, changing the drug, hospitalisation.

21. The method of claim 13, wherein responders and/or non-responders to the drug-based therapy are identified by means of an in-vitro diagnosis, characterised in that up to twelve marker sequences are selected from the group SEQ ID NO: 1-208.

22. The method of claim 13, characterised in that a remission of the rheumatoid arthritis occurs.

23. The method for managing the therapy of rheumatoid arthritis patients undergoing drug-based therapy according to claim 21, wherein the marker sequences are selected from the group
PPP5C (SEQ ID NO: 43, 147), MRPL11 (SEQ ID NO: 38, 142), TRMO (SEQ ID NO: 31, 135), RAD50 (SEQ ID NO: 45, 149), ADAMTS13 (SEQ ID NO: 27, 131), DIP2C (SEQ ID NO: 33, 137), CHD4 (SEQ ID NO: 32, 136), ALAS1 (SEQ ID NO: 29, 133), ZPR1 (SEQ ID NO: 51, 155), PDRG1 (SEQ ID NO: 41, 145)
or
MED8 (SEQ ID NO: 37, 141), HNRNPAB (SEQ ID NO: 11, 115), RBM26 (SEQ ID NO: 18, 122), UBXNI1 (SEQ ID NO: 20, 124), FAM89B (SEQ ID NO: 23, 127), TTLL12 (SEQ ID NO: 19, 123), PPP1R15A (SEQ ID NO: 17, 121), GAREM (SEQ ID NO: 25, 129), KIF5A (SEQ ID NO: 12, 116), HCLS1 (SEQ ID NO: 10, 114), ZNF579 (SEQ ID NO: 22, 126),
or
NOP58 (SEQ ID NO: 76, 180), SCFD1 (SEQ ID NO: 91, 195), MPST (SEQ ID NO: 74, 178), ILKAP (SEQ ID NO: 68, 172), ROBO1 (SEQ ID NO: 88, 192), ISCU (SEQ ID NO: 69, 173), EHBP1 (SEQ ID NO: 61, 165), PPP1R9B (SEQ ID NO: 83, 187), ZNF292 (SEQ ID NO: 103, 207), TOMM20 (SEQ ID NO: 99, 203), Clorf131 (SEQ ID NO: 54, 158),
or
SPECCIL (SEQ ID NO: 95, 199), SEC23B (SEQ ID NO: 93, 197), HDHD2 (SEQ ID NO: 67, 171, CLIP1 (SEQ ID NO: 56, 160), WDR34 (SEQ ID NO: 101, 205), EIF4G2 (SEQ ID NO: 63, 167), SSBP2 (SEQ ID NO: 96, 200), SCAP (SEQ ID NO: 90, 194), PYGB (SEQ ID NO: 85, 189), DOCK6 (SEQ ID NO: 59, 163), RFFL (SEQ ID NO: 86, 190), SUMO3 (SEQ ID NO: 97, 201).

24. A panel containing at least five marker sequences selected from the group
PPP5C (SEQ ID NO: 43, 147), MRPL11 (SEQ ID NO: 38, 142), TRMO (SEQ ID NO: 31, 135), RAD50 (SEQ ID NO: 45, 149), ADAMTS13 (SEQ ID NO: 27, 131), DIP2C (SEQ ID NO: 33, 137), CHD4 (SEQ ID NO: 32, 136), ALAS1 (SEQ ID NO: 29, 133), ZPR1 (SEQ ID NO: 51, 155), PDRG1 (SEQ ID NO: 41, 145)
or
MED8 (SEQ ID NO: 37, 141), HNRNPAB (SEQ ID NO: 11, 115), RBM26 (SEQ ID NO: 18, 122), UBXN11 (SEQ ID NO: 20, 124), FAM89B (SEQ ID NO: 23, 127), TTLL12 (SEQ ID NO: 19, 123), PPPIR15A (SEQ ID NO: 17, 121), GAREM (SEQ ID NO: 25, 129), KIF5A (SEQ ID NO: 12, 116), HCLS1 (SEQ ID NO: 10, 114), ZNF579 (SEQ ID NO: 22, 126),
or
NOP58 (SEQ ID NO: 76, 180), SCFD1 (SEQ ID NO: 91, 195), MPST (SEQ ID NO: 74, 178), ILKAP (SEQ ID NO: 68, 172), ROBO1 (SEQ ID NO: 88, 192), ISCU (SEQ ID NO: 69, 173), EHBP1 (SEQ ID NO: 61, 165), PPPIR9B (SEQ ID NO: 83, 187), ZNF292 (SEQ ID NO: 103, 207), TOMM20 (SEQ ID NO: 99, 203), Clorf131 (SEQ ID NO: 54, 158),
or
SPECCIL (SEQ ID NO: 95, 199), SEC23B (SEQ ID NO: 93, 197), HDHD2 (SEQ ID NO: 67, 171, CLIP (SEQ ID NO: 56, 160), WDR34 (SEQ ID NO: 101, 205), EIF4G2 (SEQ ID NO: 63, 167), SSBP2 (SEQ ID NO: 96, 200), SCAP (SEQ ID NO: 90, 194), PYGB (SEQ ID NO: 85, 189), DOCK6 (SEQ ID NO: 59, 163), RFFL (SEQ ID NO: 86, 190), SUMO3 (SEQ ID NO: 97, 201).
